(19) United States
(12) Patent Application Publication
(10) Pub. No.: US 2015/0164509 A1
(43) Pub. Date: Jun. 18, 2015

(54) VASCULAR COMPRESSION DEVICE

(71) Applicant: RevMedx, Inc., Wilsonville, OR (US)

(72) Inventors: John Steinbaugh, Wilsonville, OR (US); Andrew Barofsky, Lake Oswego, OR (US)

(21) Appl. No.: 14/569,477

(22) Filed: Dec. 12, 2014

Related U.S. Application Data
(60) Provisional application No. 61/915,906, filed on Dec. 13, 2013.

(51) Int. Cl. A61B 17/12

(52) U.S. Cl. CPC A61B 17/12; A61B 2017/12004

(57) ABSTRACT

Provided herein are vascular compression devices that may be used to apply pressure to an area of the body of a subject near or at a wound site following an injury or procedure (e.g., a catheterization procedure) involving one or more veins and/or arteries. The vascular compression device may include a plate having a concave face and an opposing face; a first strut and a second strut extending from and disposed adjacent opposite edges of the opposing face of the plate; and a pressure bar spaced away from the plate by the first and second struts and spanning a distance between the first and second struts.

### CROSS REFERENCE TO RELATED APPLICATIONS

The present application claims priority to U.S. Provisional Patent Application No. 61/915,906, filed Dec. 13, 2014, entitled “VASCULAR COMPRESSION DEVICE,” the disclosure of which is hereby incorporated by reference in its entirety.

### TECHNICAL FIELD

Embodiments of the present disclosure relate to the field of hemostasis, and more particularly, to vascular compression devices and methods for controlling bleeding.

### BACKGROUND

In certain circumstances, such as following a serious injury that results in heavy bleeding or an arterial procedure such as a catheterization procedure, it may be necessary to arrest bleeding (e.g., achieve hemostasis) by compressing one or more blood vessels. For example, hemostasis may be achieved by applying deep pressure with a hand or fingers and compressing one or more arteries and/or veins against a bone or other body structure.

Such compression techniques are susceptible to human error and/or human fatigue such that if pressure is not properly applied to the patient, a misdirected, misaligned, or inconsistent pressure to the artery and/or vein may prevent hemostasis from being achieved. Additionally, such a method of applying pressure requires that an individual remain with the patient, actively pressing on the wound with gloved hands directly over the artery, proximal and/or medial to the site of bleeding, for a period of time that may extend for 15-30 minutes or even longer. As a result, fatigue, stiffness, and pain may occur in the fingers, hands, wrist, and/or forearms of the practitioner performing this procedure.

### BRIEF DESCRIPTION OF THE DRAWINGS

Embodiments of the present disclosure will be readily understood by the following detailed description in conjunction with the accompanying drawings. To facilitate this description, like reference numerals designate like structural elements. Embodiments are illustrated by way of example and not by way of limitation in the figures of the accompanying drawings.

FIGS. 1A-1E illustrate a side view (FIG. 1A), end view (FIG. 1B), top view (FIG. 1C), cross sectional view (FIG. 1D), and perspective view (FIG. 1E) of one embodiment of a vascular compression device, in accordance with various embodiments;

FIGS. 2A-2C illustrate a top perspective view (FIG. 2A), a side view (FIG. 2B), and a bottom view (FIG. 2C) of a first alternate embodiment of a vascular compression device, in accordance with various embodiments;

FIGS. 3A-3C illustrate a top perspective view (FIG. 3A), a side view (FIG. 3B), and a bottom view (FIG. 3C) of a second alternate embodiment of a vascular compression device, in accordance with various embodiments;

FIGS. 4A-4C illustrate a top perspective view (FIG. 4A), a side view (FIG. 4B), and a bottom view (FIG. 4C) of a third alternate embodiment of a vascular compression device, in accordance with various embodiments;

FIGS. 5A-5C illustrate a top perspective view (FIG. 5A), a side view (FIG. 5B), and a bottom view (FIG. 5C) of a fourth alternate embodiment of a vascular compression device, in accordance with various embodiments;

FIGS. 6A-6C illustrate a top perspective view (FIG. 6A), a side view (FIG. 6B), and a bottom view (FIG. 6C) of a fifth alternate embodiment of a vascular compression device, in accordance with various embodiments;

FIGS. 7A-7C illustrate a top perspective view (FIG. 7A), a side view (FIG. 7B), and a bottom view (FIG. 7C) of a sixth alternate embodiment of a vascular compression device, in accordance with various embodiments;

FIGS. 8A-8C illustrate a top perspective view (FIG. 8A), a side view (FIG. 8B), and a bottom view (FIG. 8C) of a seventh alternate embodiment of a vascular compression device, in accordance with various embodiments;

FIGS. 9A-9C illustrate a top perspective view (FIG. 9A), a side view (FIG. 9B), and a bottom view (FIG. 9C) of an eighth alternate embodiment of a vascular compression device, in accordance with various embodiments;

FIG. 10 illustrates a top perspective view of a ninth alternate embodiment of a vascular compression device, wherein the struts are hinged to allow the struts and the pressure bar to fold flat against the concave plate, in accordance with various embodiments; and

FIG. 11 illustrates a top perspective view of a tenth alternate embodiment of a vascular compression device, in accordance with various embodiments.

### DETAILED DESCRIPTION

In the following detailed description, reference is made to the accompanying drawings which form a part hereof wherein like numerals designate like parts throughout, and in which is shown by way of illustration embodiments in which the disclosure may be practiced. It is to be understood that other embodiments may be utilized and structural or logical changes may be made without departing from the scope of the present disclosure. Therefore, the following detailed description is not to be taken in a limiting sense, and the scope of embodiments in accordance with the present disclosure is defined by the appended claims and their equivalents.

Various operations may be described as multiple discrete operations in turn, in a manner that may be helpful in understanding embodiments of the present disclosure; however, the order of description should not be construed to imply that these operations are order dependent.

The description may use perspective-based descriptions such as up/down, back/front, and top/bottom. Such descriptions are merely used to facilitate the discussion and are not intended to restrict the application of embodiments of the present disclosure.

For the purposes of the present disclosure, the phrase “A/B” means A or B. For the purposes of the present disclosure, the phrase “A and/or B” means “(A), (B), or (A and B)”. For the purposes of the present disclosure, the phrase “at least one of A, B, and C” means “(A), (B), (C), (A and B), (A and C), (B and C), or (A, B and C)”. For the purposes of the present disclosure, the phrase “(A)B” means “(B) or (AB)” that is, A is an optional element.

The description may use the phrases “in an embodiment,” or “in embodiments,” which may each refer to one or more of the same or different embodiments. Furthermore, the terms “comprising,” “including,” “having,” and the like, as used with respect to embodiments of the present disclosure, are synonymous.

Embodiments provide vascular compression devices that may be used to apply pressure to an area of the body of a subject near or at a wound site following an injury or procedure (e.g., a catheterization procedure) involving one or more veins and/or arteries. In various embodiments, the vascular compression device may enable a user to apply firm pressure to the artery and/or vein sufficient to slow or stop the bleeding, thereby achieving hemostasis. In various embodiments, the vascular compression devices disclosed herein may allow the user to apply continuous pressure to the wound using the force of his or her hand or knee, or another body part, such as a forearm, elbow, or shoulder, and such pressure may be maintained for long periods of time, such as 30 minutes or more, without fatigue to the user. Additionally, the structure of the disclosed vascular compression devices also provides stability during use, such that the vascular compression device will not slip, rotate, or become misaligned with the wound, which movement could compromise the efficacy of the vascular compression procedure.

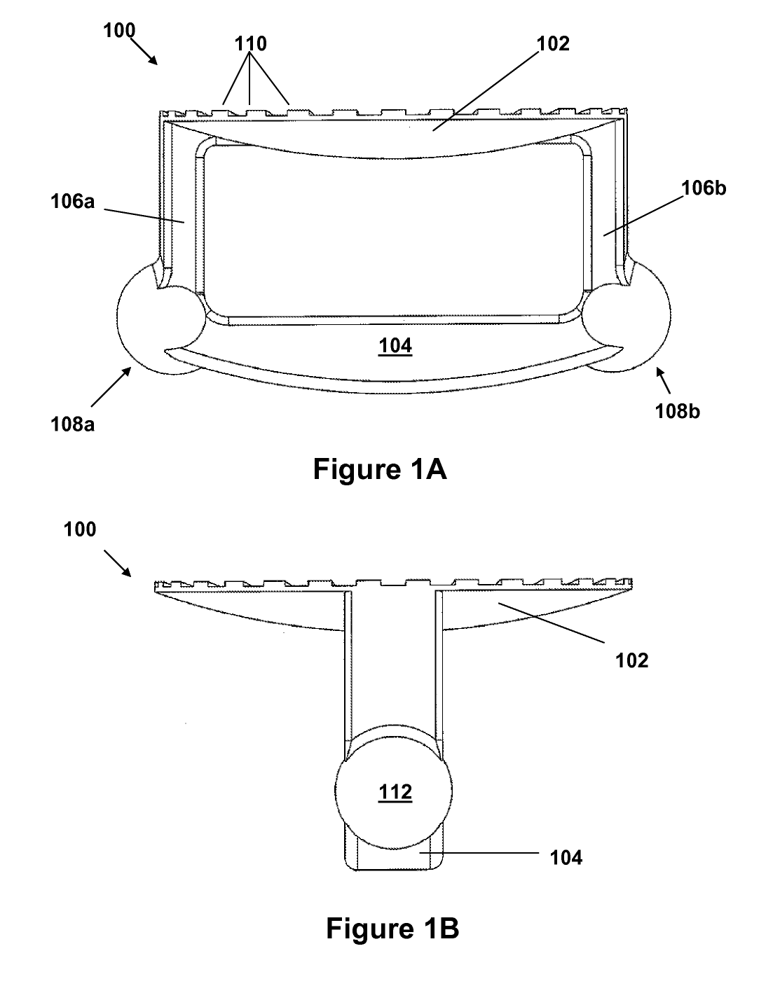

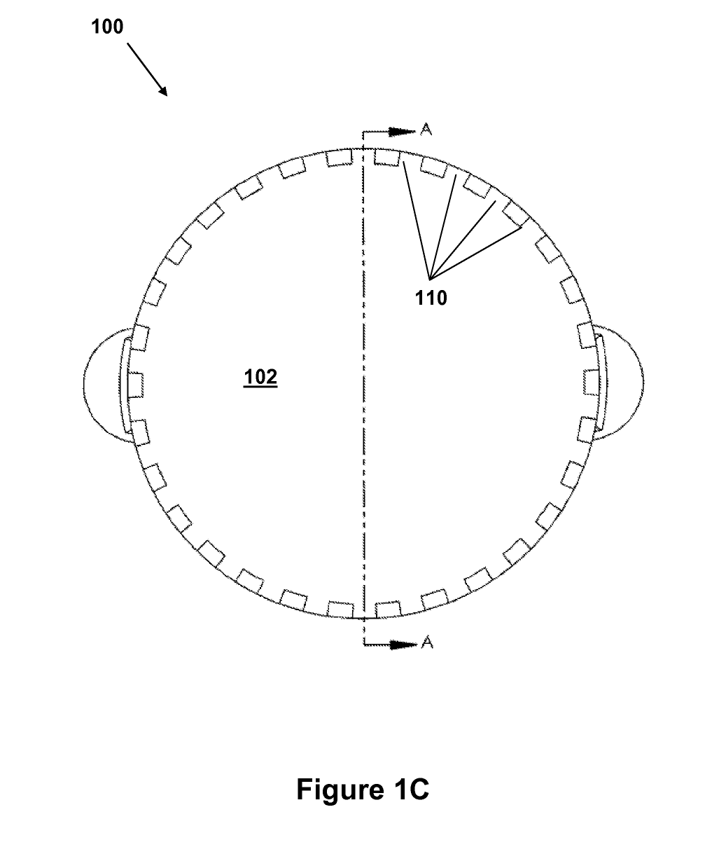

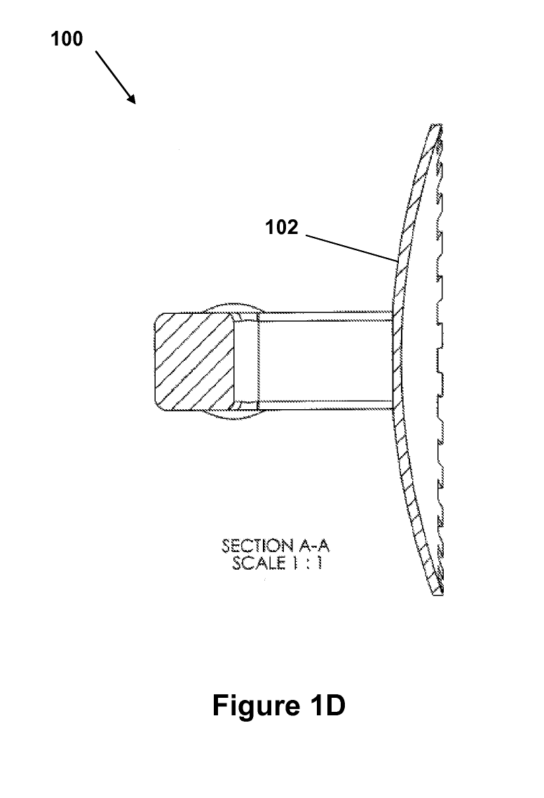

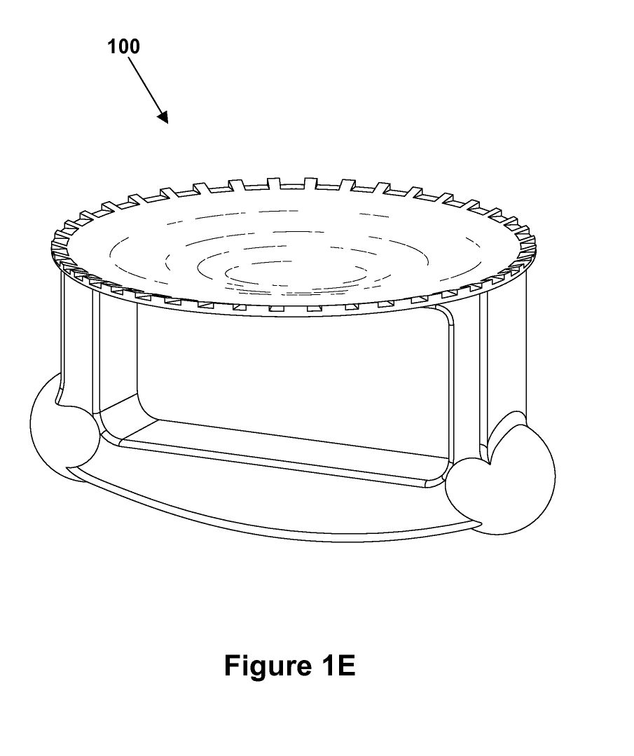

FIGS. 1A-1E illustrate a side view (FIG. 1A), end view (FIG. 1B), top view (FIG. 1C), cross sectional view (FIG. 1D), and perspective view (FIG. 1E) of one embodiment of a vascular compression device, in accordance with various embodiments. As illustrated in FIG. 1A, in various embodiments, vascular compression device 100 may include a concave plate 102 for receiving pressure from a body part of a user, a pressure bar 104 running substantially parallel to concave plate 102 and adapted to apply pressure at or near a wound on the body of a subject, and at least two struts 106a, 106b extending from concave plate 102 at or near the periphery of concave plate 102 and supporting opposing ends 108a, 108b of pressure bar 104.

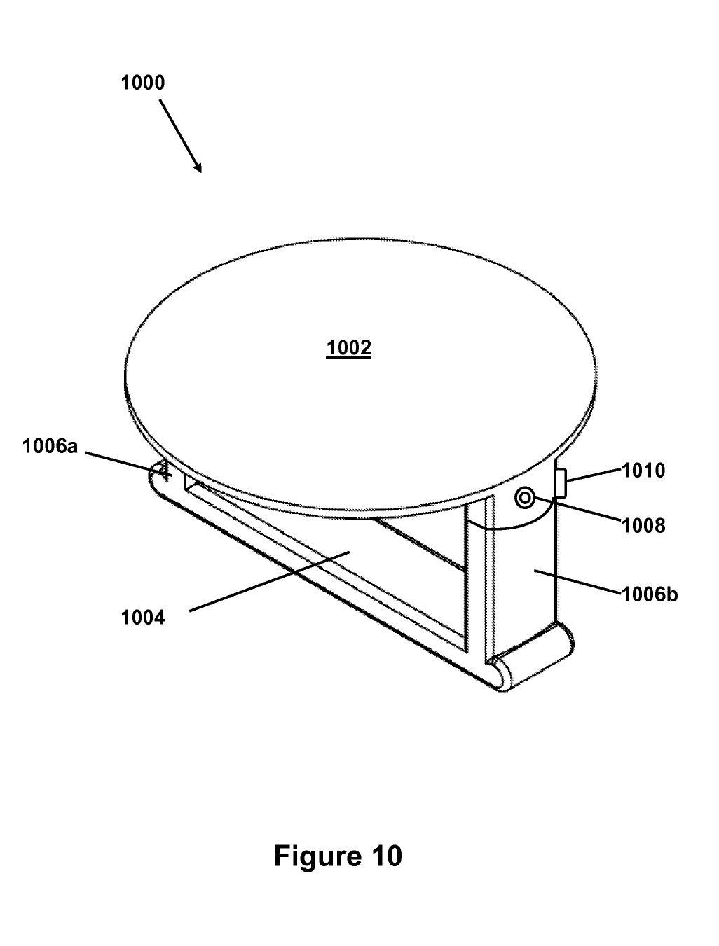

In use, in various embodiments, a user may position pressure bar 104 on a vascular pressure site, such as the femoral or subclavian artery, and may apply pressure to concave plate 102 to compress the artery and slow or stop blood flow. In various embodiments, concave plate 102 may include a concavity or recess for receiving pressure from a body part of the user, such as a hand, knee, elbow, or shoulder. The curvature of concave plate 102 can be best seen in FIG. 10. In various embodiments, the concavity or recess in concave plate 102 may prevent the knee or other body part from slipping out of position, which could occur if the plate were, instead, flat or convex. In various embodiments, such slippage could interfere with the continuous application of pressure necessary for hemostasis. In some embodiments, the concavity or recess in concave plate 102 also may help with proper projection of force to the wound site.

Turning back to FIG. 1A, in various embodiments, concave plate 102 may be round, rounded, oval, oblong, square, or any other shape suitable for receiving a knee or other body part of the user. In various embodiments, the diameter of concave plate 102 may be between about 8-15 cm, for instance about 10-13 cm or about 11-12 cm in diameter, and it may have flat edges to facilitate wrapping a compression bandage around the device to hold it in place. In some embodiments, concave plate 102 may have roughened edges or grip features 110, such as surface texture or ridges, to prevent a compression wrap from slipping. These grip features 110 in concave plate 102 also can be seen well in FIG. 1C. In some embodiments, concave plate 102 may include a non-slip pad to help prevent a knee or other body parts from slipping while applying compression.

Turning back to FIG. 1A, in various embodiments, struts 106a, 106b may have a length sufficient to apply adequate pressure on a vessel to achieve hemostasis, such as about 4-8 cm, for instance about 5-6 cm, or about 5.5 cm. In various embodiments, if struts 106a, 106b were significantly shorter, they might be unable to press pressure bar 104 sufficiently deeply to achieve hemostasis in some deep arteries, such as the femoral artery. Conversely, in various embodiments, if struts 106a, 106b were significantly longer, vascular compression device 100 may be difficult to position correctly for adequate hemostasis. In various embodiments, struts 106a, 106b may be spaced apart and may couple to and/or extend from concave plate 102 at or near the periphery or edges of concave plate 102. In some embodiments, positioning struts 106a, 106b at the periphery of concave plate 102 may add rotational stability and allow for greater control by the user in directing compressive force. One of skill in the art will appreciate that other devices, which may use only a single strut or a pair of struts positioned near the center of the device, may be likely to twist or rotate during use, which could interfere with the continuous application of pressure necessary to achieve hemostasis.

As best seen in FIG. 1B, pressure bar 104 may be rounded or radiused, or may have another shape, such as oval, rectangular, or square in profile. In some embodiments, pressure bar 104 bar may be arched, curved, or convex, such that greater pressure is applied to the vessel towards the midpoint of pressure bar 104. In various embodiments, the length of pressure bar 104 may be similar to the diameter of concave plate 102, and may span about 8-15 cm, for instance about 10-13 cm or about 11-12 cm. In some embodiments, pressure bar 104 may include one or more spherical protrusions 112 on each end. In various embodiments, these spherical protrusions 112 may be pushed into small spaces within a wound allowing the user to compress hard-to-reach arteries, such as the subclavian artery.

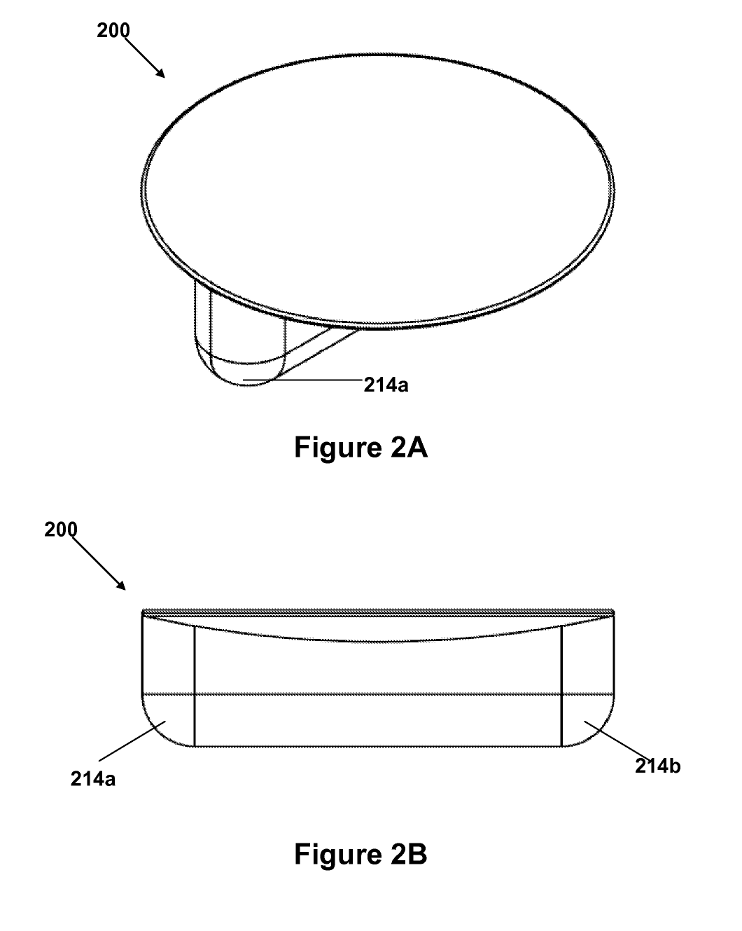

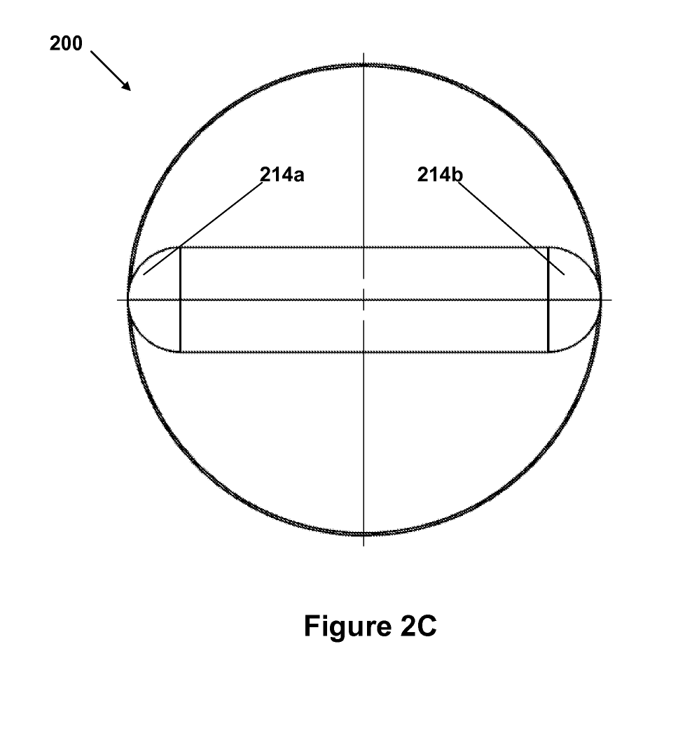

Several alternate embodiments of vascular pressure devices are depicted in FIGS. 2-9. For instance, FIGS. 2A-2C illustrate a top perspective view (FIG. 2A), a side view (FIG. 2B), and a bottom view (FIG. 2C) of an alternate embodiment of a vascular compression device 200 that lacks the spherical protrusions of the device of FIG. 1A, and instead has radiused corners 114a, 114b, in accordance with various embodiments.

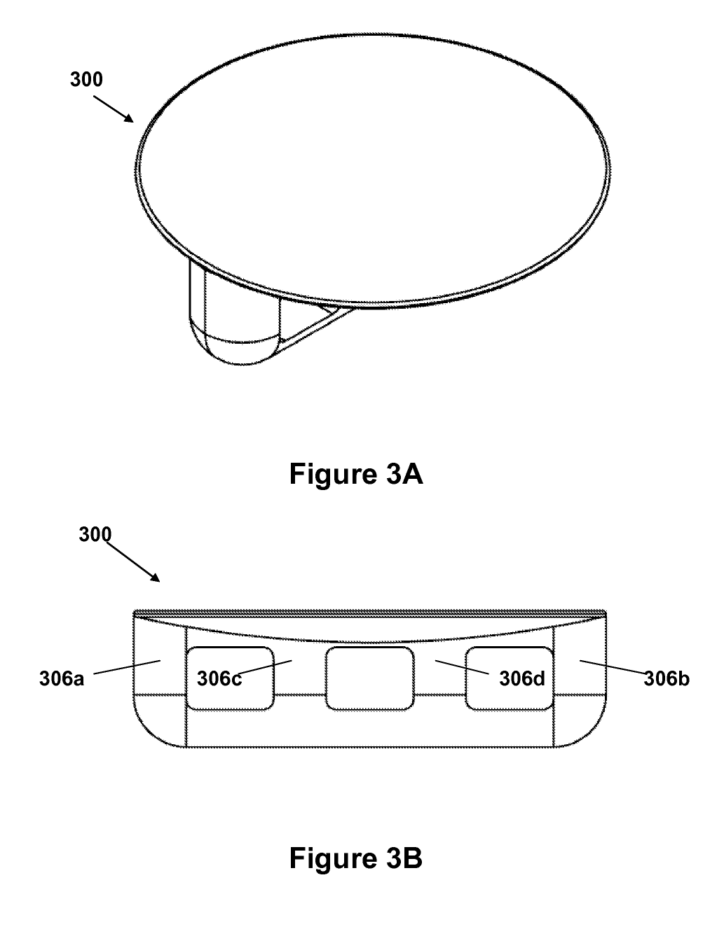

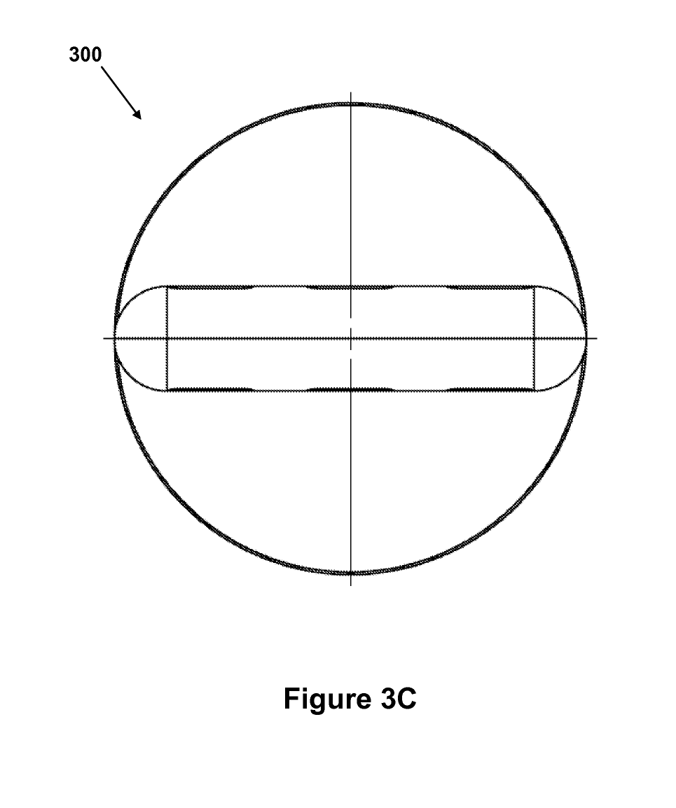

FIGS. 3A-3C illustrate a top perspective view (FIG. 3A), a side view (FIG. 3B), and a bottom view (FIG. 3C) of another alternate embodiment of a vascular compression device 300, which includes four struts 306a, 306b, 306c, and 306d (best seen in FIG. 3B), in accordance with various embodiments. Even more struts may be included in other embodiments, or the struts may be formed as a single elongated strut spanning substantially the entire width of the concave plate in still other embodiments.

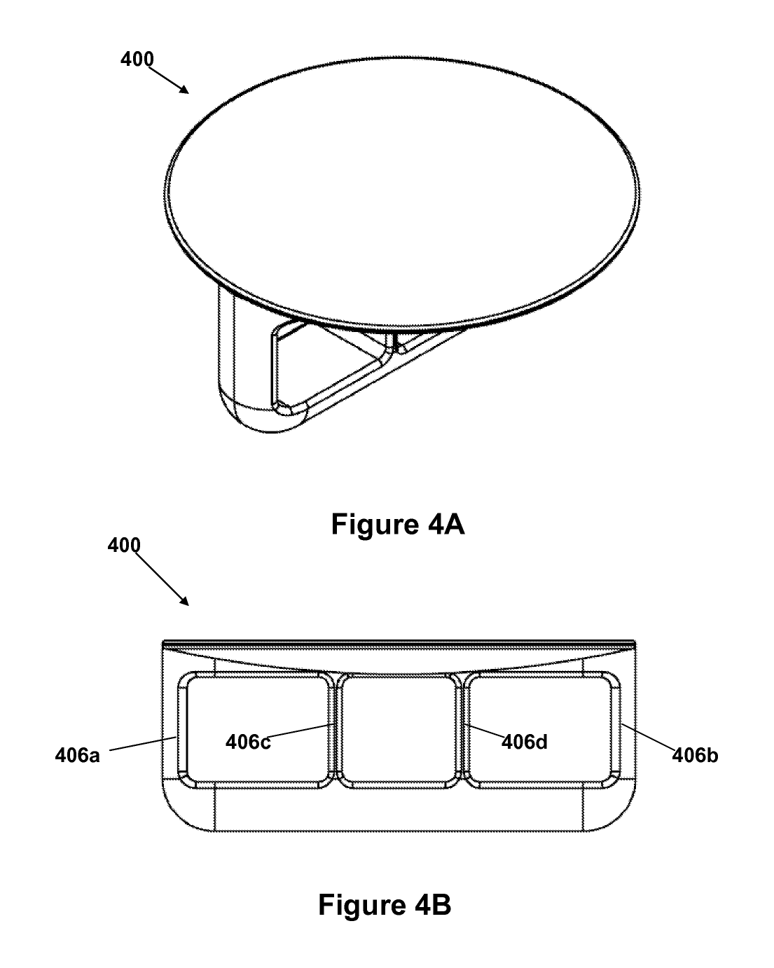

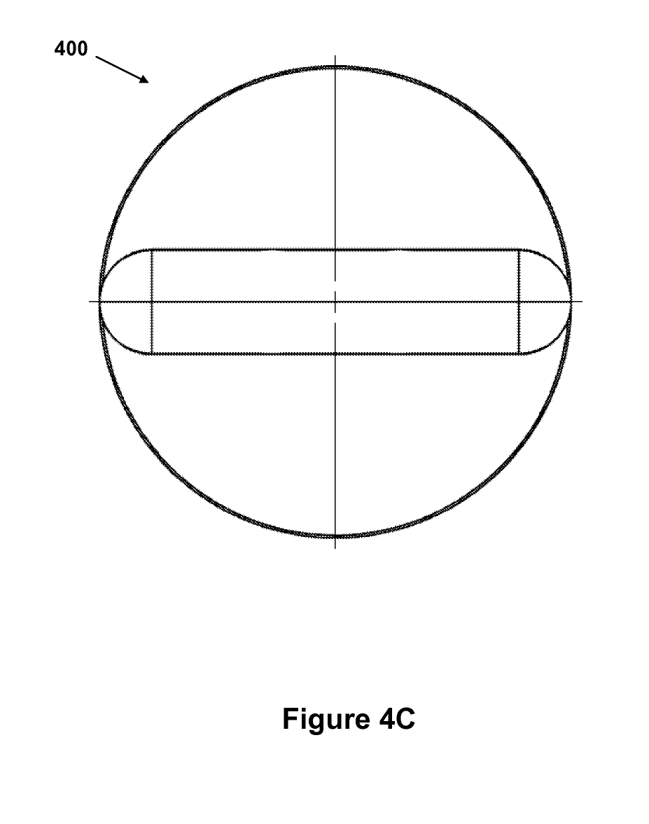

A different four-strut embodiment is illustrated in FIGS. 4A-4C, which show a top perspective view (FIG. 4A), a side view (FIG. 4B), and a bottom view (FIG. 4C) of another alternate embodiment of a vascular compression device 400, in accordance with various embodiments. Struts 406a, 406b, 406c, and 406d are best seen in FIG. 4B.

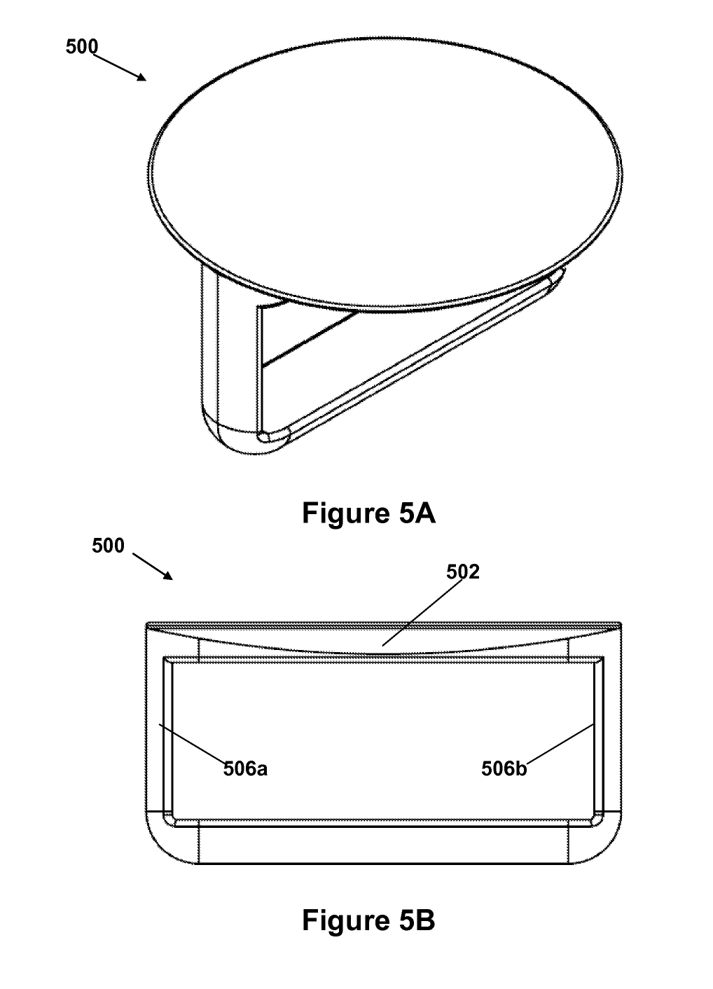

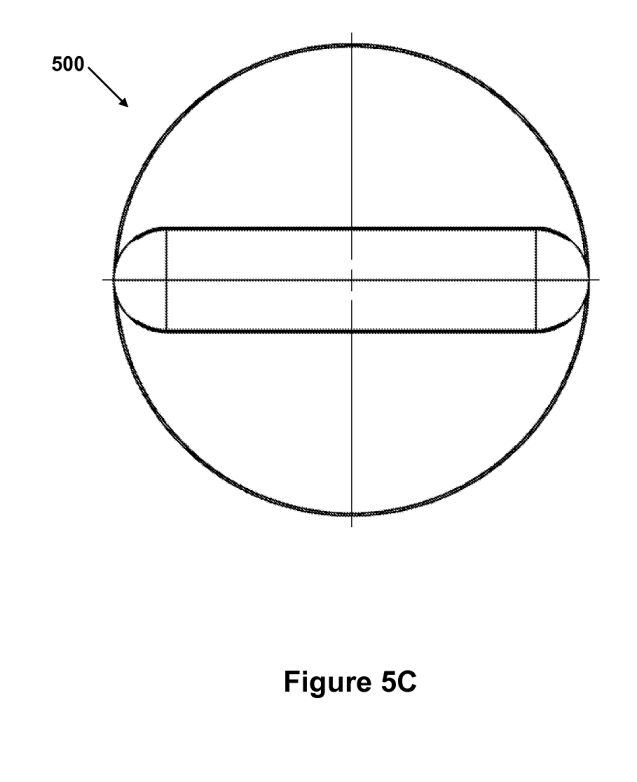

FIGS. 5A-5C illustrate a top perspective view (FIG. 5A), a side view (FIG. 5B), and a bottom view (FIG. 5C) of another alternate embodiment of a vascular compression device 500, in which the two struts 506a, 506b are positioned at the outer perimeter of concave plate 502, in accordance with various embodiments. This is best seen in FIG. 5B. In some embodiments, positioning the struts 506a, 506b at the outer edge of concave plate 502 in this fashion may enhance the lateral stability of the vascular compression device in use, and may also limit accidental rotation of the device.

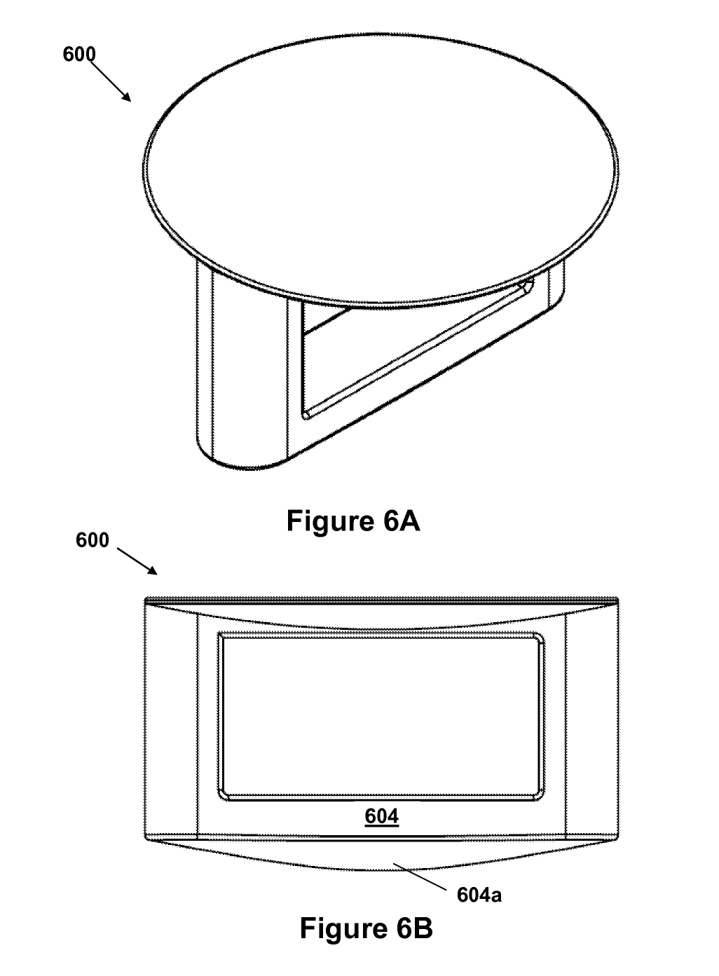

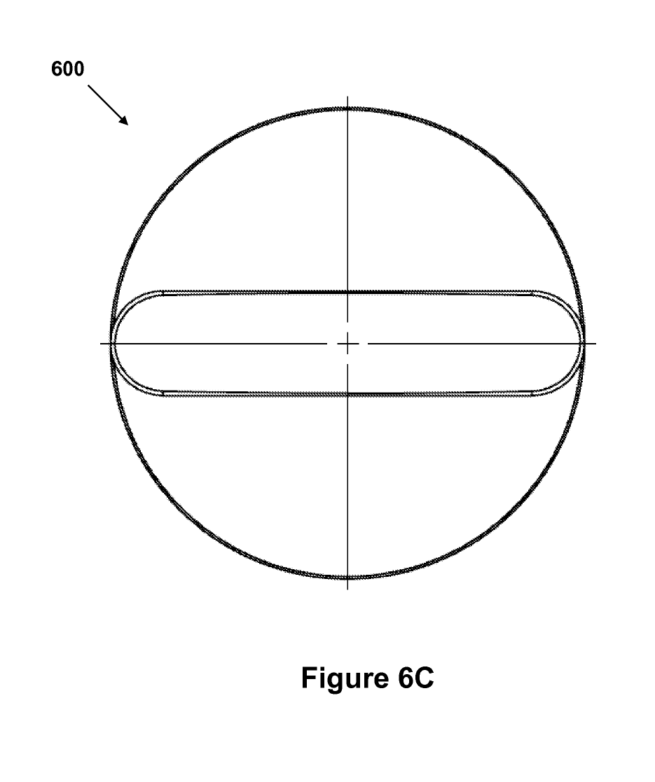

FIGS. 6A-6C illustrate a top perspective view (FIG. 6A), a side view (FIG. 6B), and a bottom view (FIG. 6C) of another alternate embodiment of a vascular compression device 600, in accordance with various embodiments. As best seen in FIG. 6B, pressure bar 604 includes a curved lower surface 604a, which may allow the vascular compression device 600 to exert more force toward the mid-point of pressure bar 604.

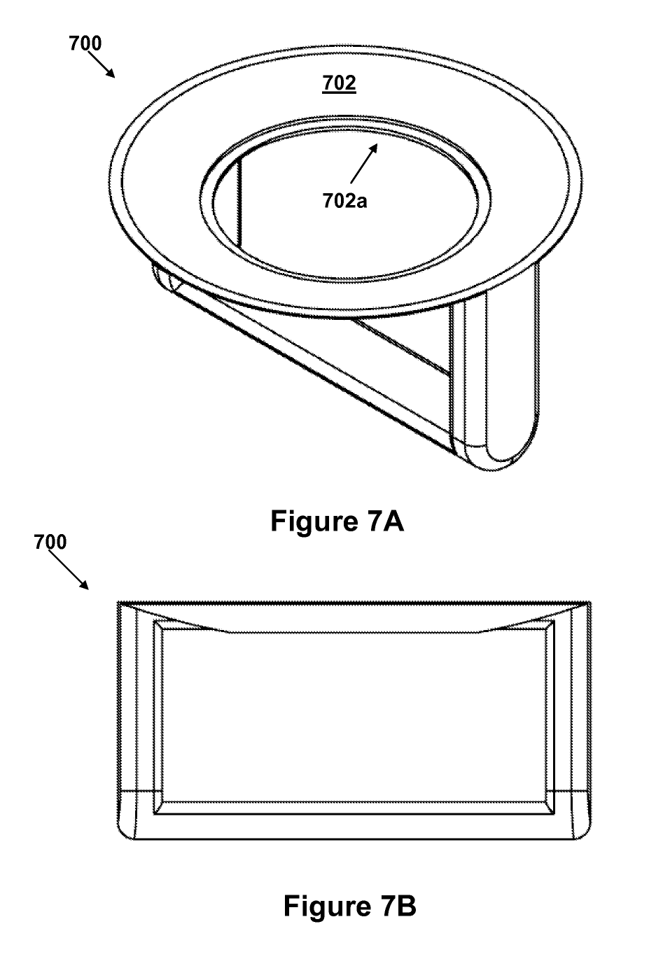

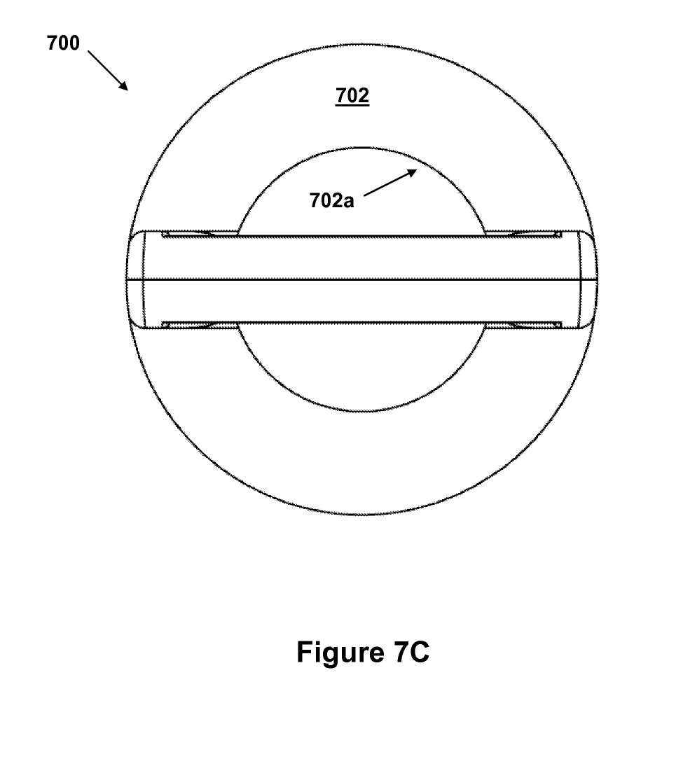

FIGS. 7A-7C illustrate a top perspective view (FIG. 7A), a side view (FIG. 7B), and a bottom view (FIG. 7C) of another alternate embodiment of a vascular compression device 700, in which the concave plate 702 includes a central cutout 702a (best seen in FIGS. 7A and 7C), in accordance with various embodiments. In various embodiments, this central cutout 702a may relieve pressure on the knee (or other body part) of the user, and it may also create a lighter vascular compression device 700. In an alternative embodiment, the cutout 702a may comprise a circular element that may be removed for another use, such as an eye protector, to enable the present device to provide multiple uses.

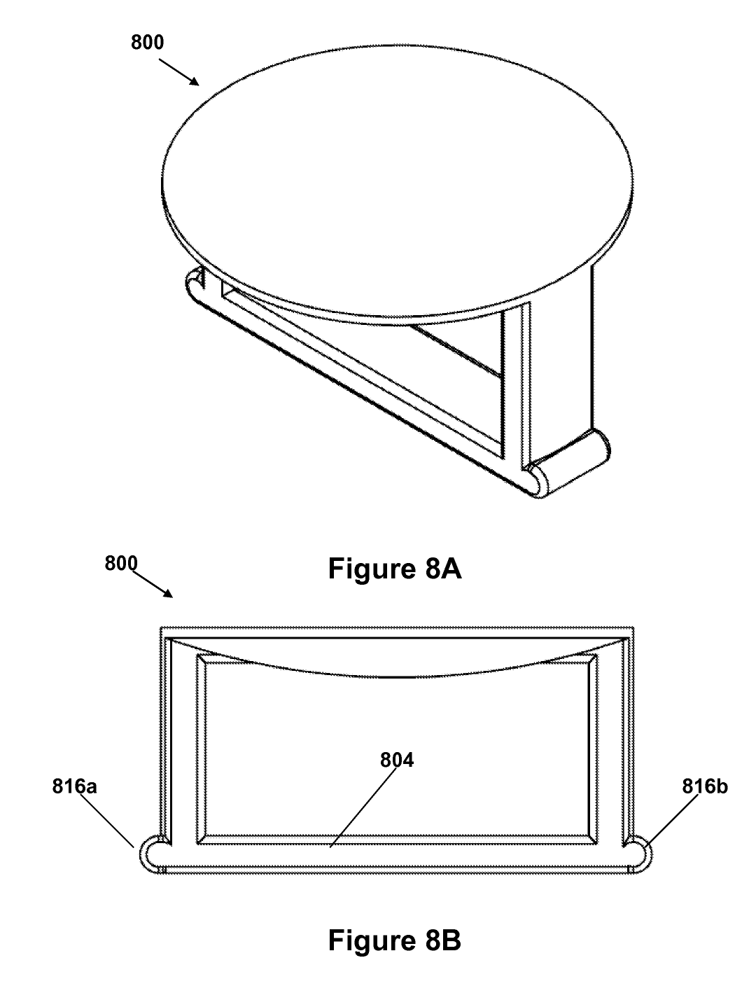

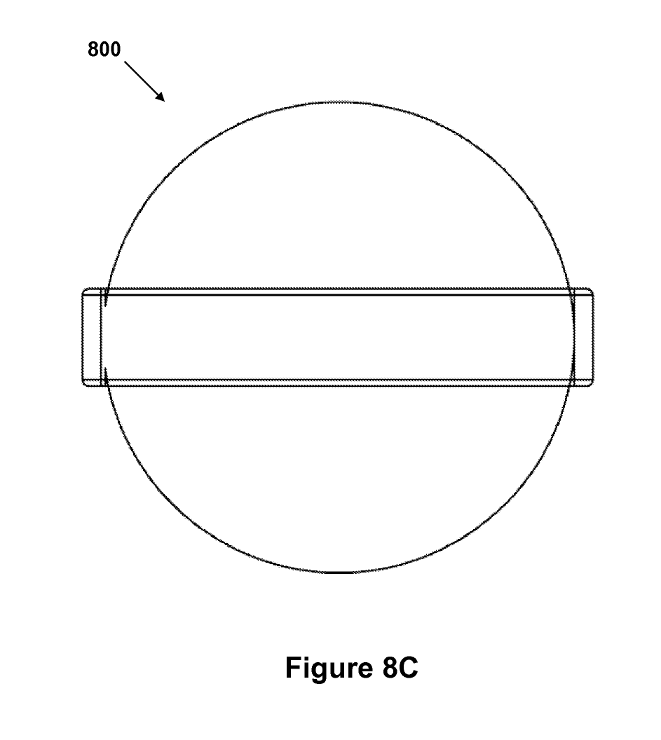

FIGS. 8A-8C illustrate a top perspective view (FIG. 8A), a side view (FIG. 8B), and a bottom view (FIG. 8C) of another alternate embodiment of a vascular compression device 800, in which the pressure bar 804 includes small radiused protrusions 816a, 816b on either end (best seen in FIG. 8B), in accordance with various embodiments. In various embodiments, radiused protrusions 816a, 816b may help a user control compression (and thereby stop blood loss) in areas of the body where there is little clearance, such as in the region surrounding the subclavian artery.

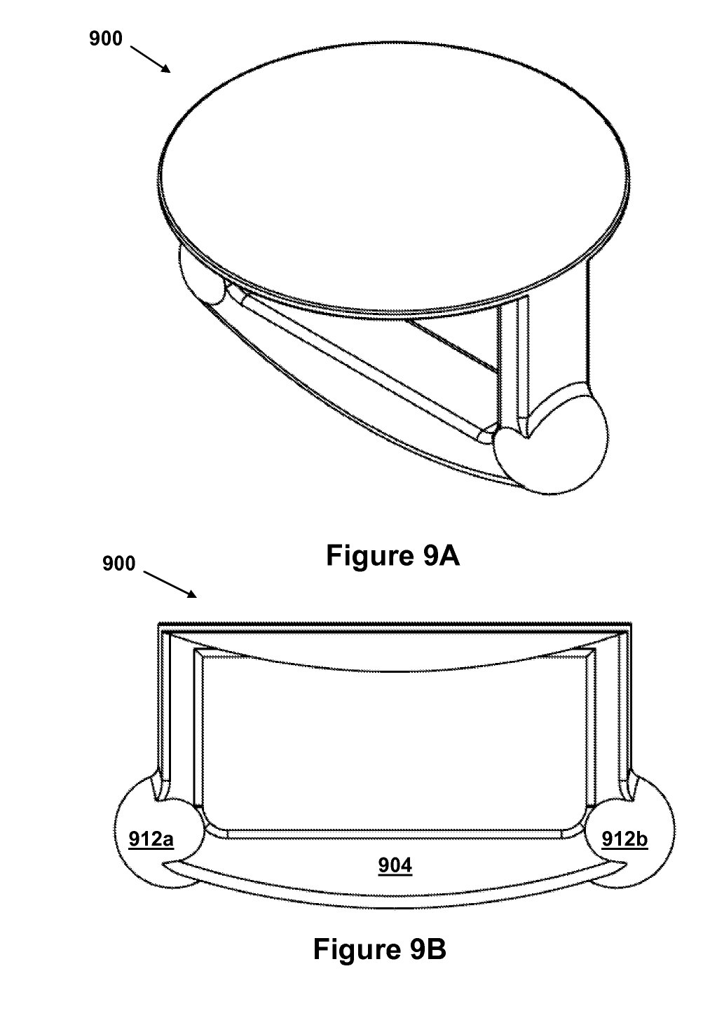

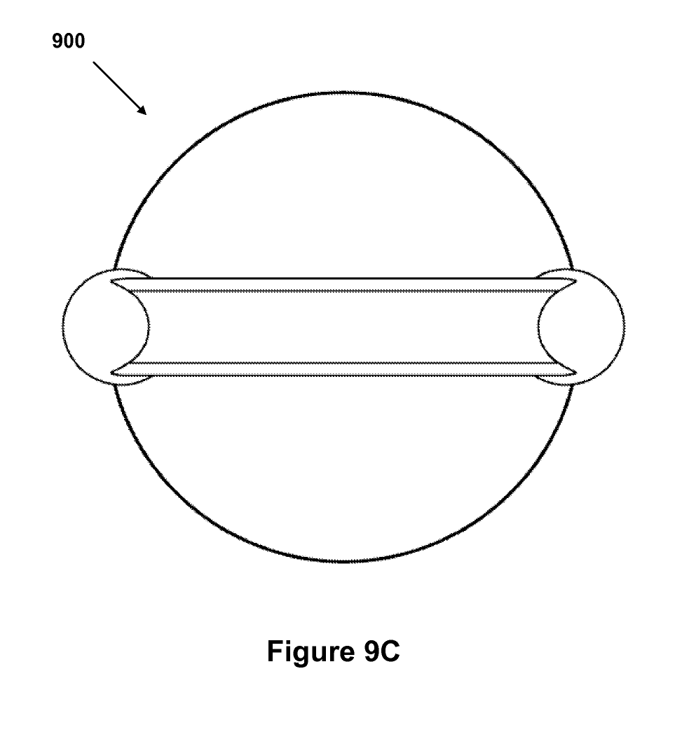

FIGS. 9A-9C illustrate a top perspective view (FIG. 9A), a side view (FIG. 9B), and a bottom view (FIG. 9C) of an eighth alternate embodiment of a vascular compression device 900, in which the pressure bar 104 is curved, with a convex curve facing the wound, and spherical protrusions 912a, 912b on either end of pressure bar 104, in accordance with various embodiments.

FIG. 10 illustrates a top perspective view of a ninth alternate embodiment of a vascular compression device 1000, in accordance with various embodiments. In the illustrated embodiment, struts 1006a, 1006b may be pivotally mounted to concave plate 1002 with one or more hinge members 1008 to enable struts 1006a, 1006b and pressure bar 1004 to be selectively positioned in a fold-flat position relative to concave plate 1002, for instance to make the device more compact when not in use. In various embodiments, the vascular compression device may include a locking mechanism 1010 to lock struts 1006a, 1006b in perpendicular position relative to concave plate 1002.

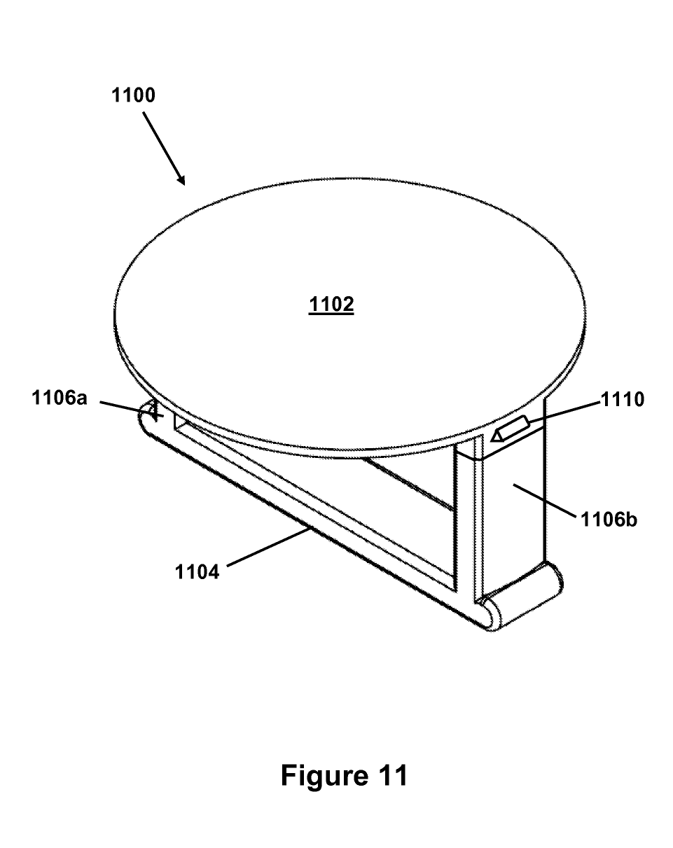

FIG. 11 illustrates a top perspective view of a tenth alternate embodiment of a vascular compression device 1100, in accordance with various embodiments. In the illustrated embodiment, struts 1106a, 1106b may be detachable from concave plate 1102 such that struts 1106a, 1106b and pressure bar 1104 may be selectively removed from concave plate 1102, for instance to make the device more compact when not in use. In various embodiments, the vascular compression device may include a locking mechanism 1110 to lock struts 1106a, 1106b in place for use once they have been coupled to concave plate 1102.

Although certain embodiments have been illustrated and described herein for purposes of description of the preferred embodiment, it will be appreciated by those of ordinary skill in the art that a wide variety of alternate and/or equivalent embodiments or implementations calculated to achieve the same purposes may be substituted for the embodiments illustrated and described without departing from the scope of the present disclosure. Those with skill in the art will readily appreciate that embodiments in accordance with the present disclosure may be implemented in a very wide variety of ways. This application is intended to cover any adaptations or variations of the embodiments discussed herein. Therefore, it is manifestly intended that embodiments in accordance with the present disclosure be limited only by the claims and the equivalents thereof.

What is claimed is:

1. A vascular compression device comprising:
a plate having a concave face and an opposing face;
a first strut and a second strut extending from and disposed adjacent opposite edges of the opposing face of the plate; and
a pressure bar spaced away from the plate by the first and second struts and spanning a distance between the first and second struts.

2. The vascular compression device of claim 1, wherein the pressure bar is substantially parallel to the plate.

3. The vascular compression device of claim 1, wherein the first and second struts extend substantially perpendicularly from the opposing face of the plate.

4. The vascular compression device of claim 1, wherein the plate has a diameter of about 8-15 cm.

5. The vascular compression device of claim 1, wherein the plate is round, rounded, oval, oblong, or square.

6. The vascular compression device of claim 1, wherein the plate comprises flat edges to enable the vascular compression device to be retained in position during use with a compression wrap.

7. The vascular compression device of claim 6, where the edges are textured to prevent the compression wrap from slipping.

8. The vascular compression device of claim 1, wherein the first and second struts each have a length of about 4-8 cm.

9. The vascular compression device of claim 1, wherein the pressure bar has a length of about 8-15 cm.

10. The vascular compression device of claim 1, wherein the pressure bar has a substantially round cross sectional profile.

11. The vascular compression device of claim 1, wherein the pressure bar has a substantially square cross sectional profile.

12. The vascular compression device of claim 11, wherein the substantially square cross sectional profile has radiused corners.

13. The vascular compression device of claim 1, wherein the pressure bar comprises an arch.

14. The vascular compression device of claim 13, wherein an apex of the arch faces away from the plate.

15. The vascular compression device of claim 1, wherein the pressure bar has a convex side facing away from the plate.

16. The vascular compression device of claim 1, wherein the pressure bar comprises a first end and a second end, and wherein the pressure bar comprises a spherical protrusion at each of the first and second ends.

17. The vascular compression device of claim 1, wherein the pressure bar forms a radiused corner with each of the first and second struts.

18. The vascular compression device of claim 1, wherein each of the first and second struts is hingeably coupled to the plate such that the pressure bar and at least a portion of each of the first and second struts may be selectively folded towards the plate.

19. The vascular compression device of claim 18, wherein the device further comprises a locking mechanism to selectively retain the first and second struts in an extended position.

20. A vascular compression device comprising:
a plate having a concave face and an opposing face; and
a pressure bar extending from the opposing face of the plate and extending substantially across the opposing face.
